(19) United States
(12) Patent Application Publication
(10) Pub. No.: US 2014/0074161 A1
(43) Pub. Date: Mar. 13, 2014

(54) WOUND CLOSURE DEVICES AND METHODS OF USE

(71) Applicant: MimOSA Medical, Inc., Menlo Park, CA (US)

(72) Inventors: Michael H. ROSENTHAL, Menlo Park, CA (US); Donald A. GONZALES, San Antonio, TX (US)

(73) Assignee: MimOSA Medical, Inc., Menlo Park, CA (US)

(21) Appl. No.: 13/839,199

(22) Filed: Mar. 15, 2013

Related U.S. Application Data
(60) Provisional application No. 61/698,279, filed on Sep. 7, 2012.
(60) Provisional application No. 61/710,516, filed on Oct. 5, 2012.

(51) Int. Cl. A61B 17/04

(52) U.S. Cl. CPC A61B 17/0401
USPC 606/232

(57) ABSTRACT

Wound closure devices and methods of their use are described herein. A deployment instrument may be used to deploy and position tissue anchors which may be locked or secured along lengths of suture in suturing and plicating soft tissues, particularly along tissue regions located in areas of the body where space is limited. Generally, the fastening device may comprise a housing having one or more adjustable tissue anchors aligned longitudinally, wherein each of the one or more adjustable tissue anchors defines a receiving channel along a periphery of the tissue anchors, a tether having a length which is positionable within the receiving channel of each of the tissue anchors, and a plunger translatable relative to the housing, where movement of the plunger urges the one or more tissue anchors to attach securely to the tether.

### CROSS-REFERENCE TO RELATED APPLICATIONS

This application claims the benefit of priority to U.S. Prov. Apps. 61/698,279 filed Sep. 7, 2012 and 61/710,516 filed Oct. 5, 2012, each of which is incorporated herein by reference in its entirety.

### FIELD OF THE INVENTION

The present invention relates generally to medical devices used for securing and approximating wounds and tissue regions. More particularly, the present invention relates to apparatus and methods for approximating wounds and tissue regions towards one another for wound repair and/or securing portions of tissue for various treatments.

### BACKGROUND OF THE INVENTION

Suturing deep tissues and performing suturing techniques in confined spaces remains a difficult challenge. The ability to tie knots requires a two-handed technique and is fraught with challenges especially when using endoscopic and/or laparoscopic instruments for treating, e.g., oropharyngeal tissues, or regions of the body where space constraints or exposed knots are an issue.

In addition to the difficulty in performing such techniques, the treatment outcomes are often less than ideal. For instance, when closing the oropharyngeal tissue with suture such as for an uvulopalatopharyngoplasty (UPPP) procedure, the suture typically tends to pull through the friable mucosa and may result in pain inflicted upon the patient due to the exposed or extruded suture knots. Additionally, the time required can often exceed 20 minutes for the closure. The same problems are often seen in other procedures performed upon mucosal tissues such as for bowel anastomosis, closure of biopsy wounds, plications of the stomach, etc. This phenomenon is not only limited to mucosal tissue but also other areas where soft tissues are approximated or secured, such as for shoulder plications, etc.

Thus, there is a need for instruments and procedures which allow for the deployment of tissue securement devices which enable the securement of soft tissue regions, particularly in areas of the body where space is limited.

### BRIEF SUMMARY OF THE INVENTION

A deployment instrument which deploys and positions tissue anchors which may be locked or secured along lengths of suture may be used in suturing and plicating soft tissues, particularly along tissue regions located in areas of the body where space is limited (e.g., gastrointestinal tissues, oropharyngeal, laryngeal, vascular tissues, etc.). The variations of the tissue anchors described herein may also function to lock against suture lengths without the need for tying knots or exposing terminal suture lengths which typically produce negative clinical outcomes. The instruments and tissue anchors may also produce tissue closures with reduced procedure times, create more durable closures, and create improved patient outcomes with reduced pain and improved healing.

Generally, the fastening device may comprise a housing having one or more adjustable tissue anchors aligned longitudinally, wherein each of the one or more adjustable tissue anchors defines a receiving channel along a periphery of the tissue anchors, a tether having a length which is positionable within the receiving channel of each of the tissue anchors, and a plunger translatable relative to the housing, where movement of the plunger urges the one or more tissue anchors to attach securely to the tether.

The one or more tissue anchors may be aligned within an anchor cartridge which may be attached to the housing. With this deployment instrument, one example of a method for fastening tissue may generally comprise passing a tether through a first portion of tissue until a first anchor attached to the tether is placed against the first portion of tissue, passing the tether through a second portion of tissue, aligning a second anchor with the tether, and tensioning the first anchor and the second anchor relative to one another such that the first and second portions of tissue are approximated towards one another. With the approximated tissue, they may then be maintained by securing the second anchor to the tether such that the first and second portions of tissue remain secured to one another.

### BRIEF DESCRIPTION OF THE DRAWINGS

FIGS. 1A-1C shows an example of a UPPP procedure where the oropharyngeal tissue at the back of the throat is typically localized and sutured.

FIG. 2A shows a perspective view of one variation of a single pair of locking anchors (or caps) which are tethered to one another through plicated tissue via a length of suture extending between the anchors and through the approximated tissue.

FIG. 2B shows a perspective view of another variation of multiple adjacent locking anchors secured to a corresponding anchor through a length of plicated tissue.

FIGS. 3A to 3C show top and perspective views of another variation of locking anchors which are rotatable relative to one another to secure a length of suture or a tether within the anchor.

FIGS. 4A to 4D show an example of how a length of suture may be introduced along a side window of a rotatable anchor and secured.

FIG. 5 shows an example of a Fibonacci curve or spiral which may be reproduced along a profile of a locking window of rotatable anchor.

FIGS. 6A to 6D show perspective and top views of a locking anchor variation having a Fibonacci spiral profile which allows the anchor to secure itself to variable diameter suture lengths.

FIGS. 7A to 7E show top views of the locking anchor of FIG. 6A illustrating how a suture length may be loaded along a side window and secured to the anchor by the relative rotation of the anchor portions.

FIGS. 8A and 8B show perspective and exploded assembly views of another variation of a locking anchor utilizing a cam mechanism to secure a suture to the anchor.

FIGS. 9A to 9D illustrate an example of how the anchor of FIG. 8A may introduce a suture length along a side channel of the anchor and subsequently locked via a cam mechanism.

FIGS. 10A to 10C show top views of the anchor of FIG. 8A illustrating the introduction of the suture along the side channel of the anchor and locking of the suture via the bearing cam.

FIG. 11 shows a perspective and detail perspective view of one variation of a deployment instrument utilizing an anchor cartridge.

FIGS. 12A to 12C show partial cross-sectional side views of a variation of the deployment instrument illustrating the anchor cartridge and optional tissue grasper integrated with the instrument.

FIGS. 13A and 13B show cross-sectional side views of the tissue grasper which may be configured to receive an anchor.

FIG. 14A shows a perspective view of another variation of the deployment instrument having a hooked grasping arm.

FIGS. 14B and 14C show side views of the instrument of FIG. 14A illustrating how the hooked grasping arm may be actuated relative to the instrument housing.

FIGS. 15A and 15B show side views of another variation of the deployment instrument having a receiving window or channel along the instrument housing.

FIGS. 16A and 16B show side views of yet another variation of the deployment instrument having a V-shaped groove receiving channel actuatable relative to the instrument housing.

FIGS. 17A and 17B show detail cross-sectional side views of how a pair of locking anchors may be staged for deployment from the anchor cartridge.

FIGS. 18A to 18I illustrate an example of how the pair of locking anchors may be staged from the anchor cartridge and subsequently ejected from the deployment instrument for securement to the tissue region of interest.

FIG. 19 shows an assembly view of another variation of a deployment instrument with a pair of locking anchors and a suture length attached to a suturing needle prior to deployment in the tissue region.

FIG. 20 shows a perspective view of the deployment instrument of FIG. 19.

FIGS. 21A to 21C show detail side and perspective views of the deployment instrument of FIG. 19 illustrating how a plunging portion may be moved relative to an anvil portion to secure an anchor to the tissue region.

FIG. 22 shows a perspective view illustrating how a suture length attached to a deployed locking anchor secured to a first portion of tissue may be attached to the deployment instrument.

FIGS. 23A to 23H show another variation of how a first locking anchor may be secured to the first portion of tissue and tightened to a second corresponding locking anchor secured to a second portion of tissue via the deployment instrument and subsequently tightened to approximate the first and second portions of tissue.

### DETAILED DESCRIPTION OF THE INVENTION

In suturing and plicating soft tissues, particularly along tissue regions located in areas of the body where space is limited (e.g., gastrointestinal tissues, oropharyngeal, laryngeal, vascular tissues, etc.) a deployment instrument which deploys and positions tissue anchors which may be locked or secured along lengths of suture may be used. The variations of the tissue anchors described herein may also function to lock against suture lengths without the need for tying knots or exposing terminal suture lengths which typically produce negative clinical outcomes. The instruments and tissue anchors may also produce tissue closures with reduced procedure times, create more durable closures, and create improved patient outcomes with reduced pain and improved healing.

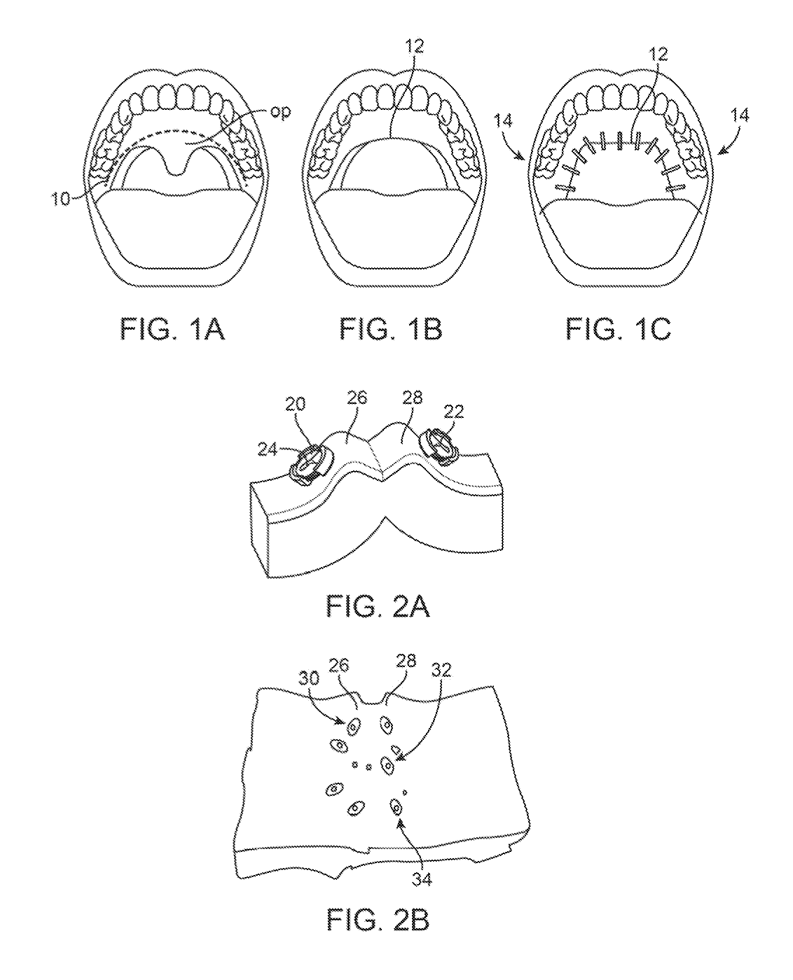

FIGS. 1A-1C shows an example of a UPPP procedure where the oropharyngeal tissue OP at the back of the throat is typically localized and sutured. An example of how such tissue OP may be resected is illustrated by the line of resection 10 along which the resected tissue 12 may be removed to enlarge the passageway. However, once the resected tissue 12 is approximated or removed, one or more sutures 14 are typically used to approximate the resected edges of the tissue towards one another to facilitate healing of the wound. Such a procedure provides an example of one application for the instruments and anchors described herein. The apparatus and methods described herein may thus be used in a variety of regions and for various procedures. For example, aside from closing the tonsillar pillars in a UPPP procedure, the anchors and instruments may be used in other procedures such as dental procedures such as tooth extractions, surgeries involving the gastrointestinal and genitourinary systems, etc.

FIG. 2A shows a perspective view illustrating how a single pair of tissue anchors may have each corresponding anchor locked to a length of suture, wire, or a tether and passed through two approximated tissue regions and terminating at each anchor. In this example, a first anchor 20 may be secured to a first end of the suture or tether 24 and secured near or at a first tissue region 26 to be approximated. The suture or tether 24 may be passed through the tissue such that the first anchor 20 acts as a stop against the tissue surface to allow the first tissue region 26 to be pulled towards the second tissue region 28. The suture or tether 24 may also be passed through the second tissue region 28 and secured along the second anchor 22. The first and second anchors 20, 22 may be approximated towards one another while the first or second anchor 20, 22 slides along the suture or tether 24 until the edges of the first and second tissue regions 26, 28 are drawn towards one another into contact to facilitate healing between the tissue regions 26, 28. With the anchors 20, 22 and respective tissue regions 26, 28 desirably positioned, the suture or tether 24 may be tensioned and secured to the anchors 20, 22 to maintain the position of the tissue. Because the tissue anchors 20, 22 may provide a relatively widened contact surface against the tissue surfaces, the threes imparted upon the respective tissue regions 26, 28 may be distributed over the surfaces of the anchors 20, 22 and prevent or inhibit the anchors 20, 22 from pulling through or extruding through the tissue.

FIG. 2B shows a perspective view of another example of how multiple pairs of corresponding tissue anchor assemblies may be positioned adjacent to one another to secure a length of tissue. With the respective first and second tissue regions 26, 28 shown in apposition to one another, distinct or separate anchor pairs are illustrated securing the tissue together. For instance, a first anchor pair 30, a second anchor pair 32, and a third anchor pair 34 are illustrated extending between the tissue regions 26, 28 adjacent to one another in an interrupted suture pattern. However, other variations may utilize a continuous suture or tether along the length of the tissue regions 26, 28.

Locking Anchor Devices

The tissue anchors described herein may be deployed and secured to one or more tissue regions utilizing the deployment instruments also described herein. As such, variations of the tissue anchors or caps are now described.

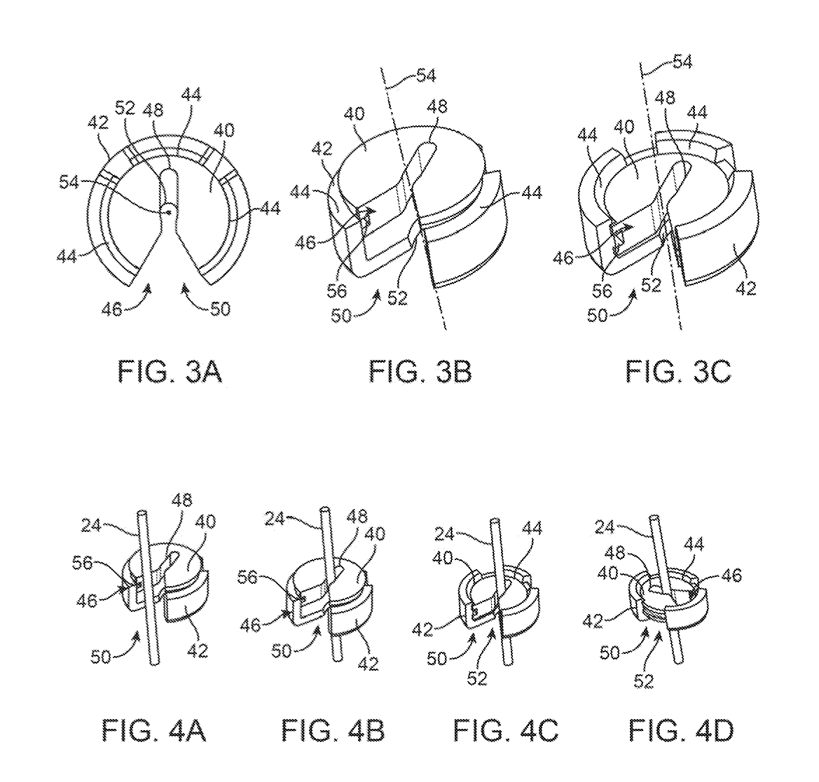

One variation is shown in the top and perspective views of FIGS. 3A to 3C which illustrate a rotational locking mechanism. In this suture side loading variation, this design includes two components; an inner crimping disc 40 and an outer housing 42. In this variation, the crimping disc 40 and outer housing 42 are each formed into a circular configuration to facilitate respective rotation of the components. The inner crimping disc 40 may optionally define a peripheral slot 56 around a circumference of the disc 40 into which a retaining lip 44 defined by the outer housing 42 may reside, as shown in the perspective view of FIG. 3B. Prior to deployment and loading a suture or tether into the anchor, the crimping disc 40 may be rotatably positioned to have peripheral slot 56 of disc 40 aligned with retaining lip 44 such that the inner crimping disc 40 may be partially snapped into the outer housing 42 to form a single assembly, as shown in the perspective view of FIG. 3C.

The outer housing 42 component may be constructed from materials or dimensions that cause it to be less flexible and/or malleable than the inner crimping disc 40. A slot in the side of both components communicates from the circumference to at least the center line 54. For instance, inner crimping disc 40 may define a disc opening 46 which tapers radially towards centerline 54 to form a disc slot 48 for receiving a suture or tether. Likewise, outer housing 42 may also define a housing opening 50 which tapers radially towards centerline 54 to form a housing slot 52. These respective openings 46, 50 may include a mouth with an opening substantially larger than the suture or tether diameter and tapers to their respective slots 48, 52 width similar in size to the tether diameter, e.g., 0.004 in. to 0.020 in. The slots 48, 52 in the assembly provide the advantage of allowing the suture or tether to engage into the anchor without passing through a hole in the assembly. That is, because the openings 46, 50 are defined along a periphery of the anchor assembly, the anchor may engage the suture or tether at any point along its length. Additionally, it is not a requirement for the potentially relatively rigid and large diameter tissue piercing component of the closure system to travel through the assembly to mate the suture or tether and anchor.

FIGS. 4A to 4D illustrate an example of how the locking anchor may engage the suture or tether and are locked in place. With the crimping disc 40 positioned about the retaining lip 44 of outer housing 42, suture or tether 24 may be introduced through the respective openings 46, 50 which are initially aligned with one another, as shown in FIG. 4A, until the suture or tether 24 is positioned along the slots 48, 52, as shown in FIG. 4B. The two components may be compressed and snapped together in the compressed state mainly in the axial direction such that crimping disc 40 is pressed within outer housing 42 and secured via retaining lip 44, as shown in FIG. 4C. The interference fit between the inner crimping disc 40 outer diameter and the outer housing 52 inner diameter may cause the inner crimping disc slot 48 to collapse at least partially as the parts mate. The collapsing inner slot 48 may crimp onto the suture or tether fixing the anchor assembly to the suture or tether. Each of the components to be designed to rotate relative to one another as shown. For example as shown in FIG. 4D, the crimping disc 40 may be rotated at least partially relative to outer housing 42 such that the openings 46, 50 are moved out of alignment with one another to ensure that the suture or tether is confined in the centerline 54 of the anchor assembly.

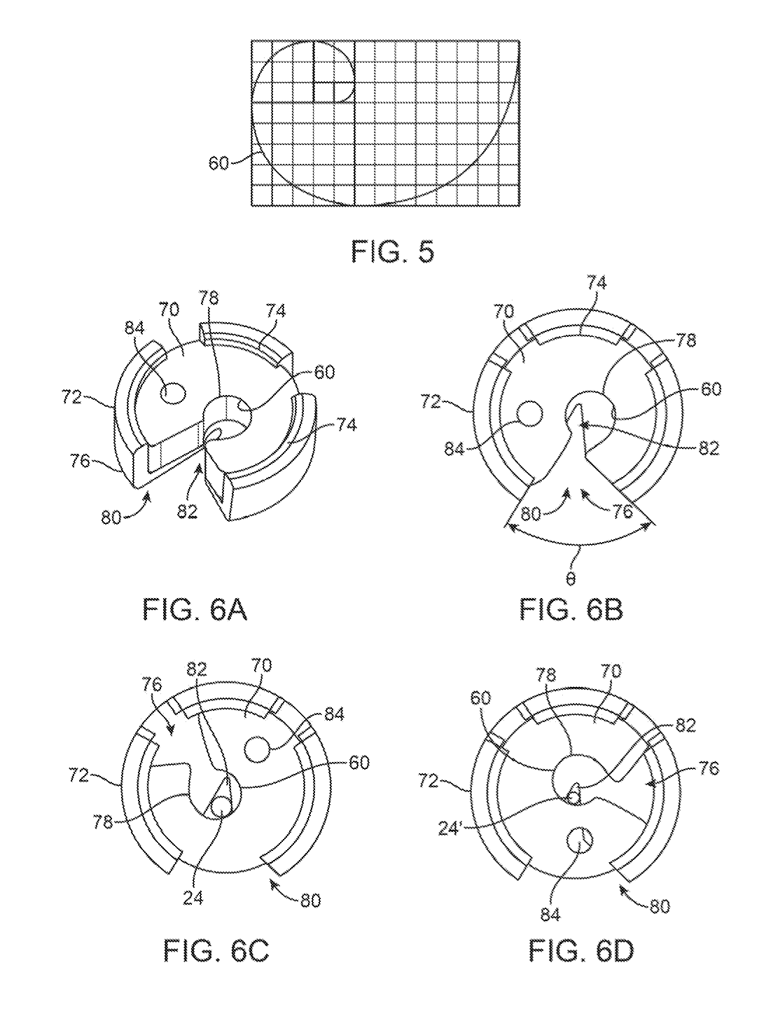

An alternative rotational locking mechanism is illustrated in the perspective and top views of FIG. 6A to 6D. The variation shown may utilize a spiral or curved cinching mechanism which may utilize a cap having spiraled or curved profile, e.g., a Fibonacci spiral or curve 60, as shown in FIG. 5. Such a variation may accommodate a wide range of suture or tether diameters through a graded relative rotation between components of the locking anchor. Also, such a locking anchor may be readily unlocked and removed from the suture, if desired. As with the previous variation, an inner crimping disc 70 may be rotatably positioned within an outer housing 72 while secured via retaining lip 74. The inner crimping disc 70 may define a disc opening 76 which narrows to a disc slot 78 which may be defined by the Fibonacci spiral or curve 60. The outer housing 72 may likewise define a housing opening 80 which tapers to a housing slot 82.

As shown, the suture or tether 24 may slide into the anchor through the disc opening 76 and housing opening 80 side slots when aligned with one another. The openings 76, 80 may range over opening angle Θ which may be varied to accommodate any number of different size sutures or tethers 24. In this variation, the housing slot 82 may be slightly smaller in width than the suture or tether 24 outer diameter and include a bottom face geometry that ensures the suture or tether 24 stays engaged in the assembly preferentially opposite the opening 80. This configuration may provide for the suture or tether 24 to freely move within the anchor prior to the locking step. This may also accommodate any external lateral forces being applied to the suture or tether 24 without preferentially forcing the suture or tether 24 to disengage from the assembly. The outer housing 72 may be tapered and sized to accept sutures or tethers having diameters of up to 2-0 (e.g., 0.3-0.35 mm), as shown by suture 24 in FIG. 6C, and down to 5-0 (e.g., 0.1-0.15 mm), as shown by suture 24′ in FIG. 6D.

As described, the disc slot 78 may define a spiral or partial spiral profile along the face that engages the suture tether. A partial spiral may be modeled, e.g., using the Fibonacci spiral or curve 60 to ensure that a force vector normal to the main axis of the outer housing 72 housing slot 82 may be achieved. This force vector direction may ensure secure engagement of the suture or tether 24 in the depth of the cinching slot prior to pinching or approximating between the components.

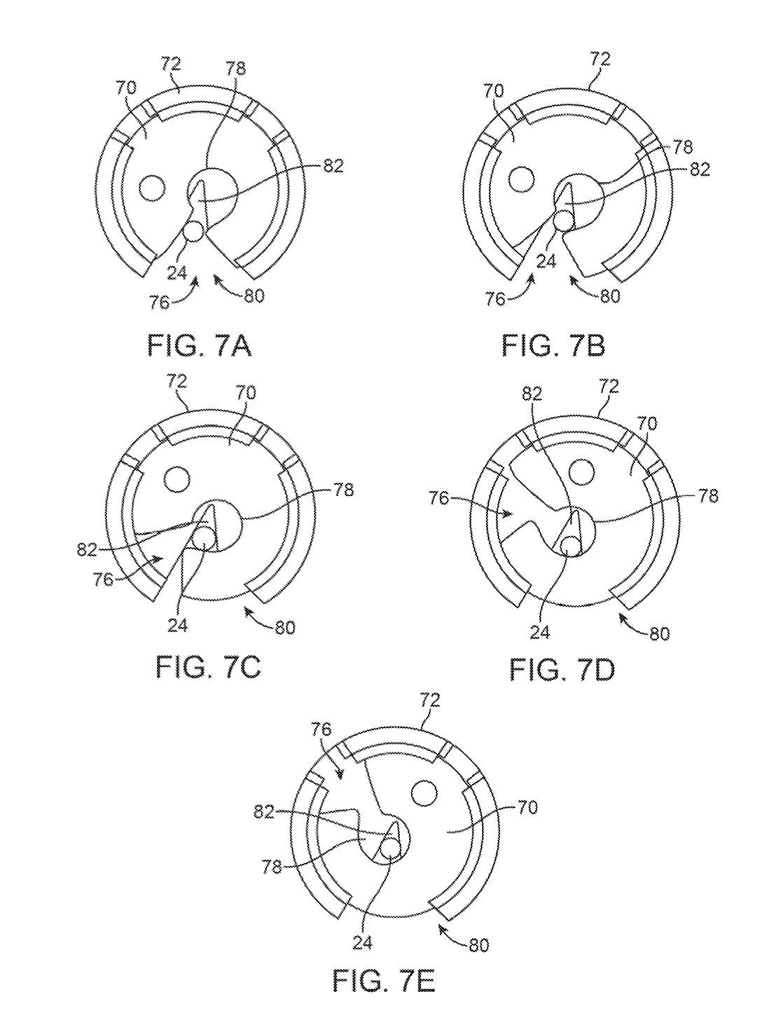

An example is shown in the top views of FIGS. 7A to 7E. Initially, the slots 76, 80 may be aligned to allow the suture or tether 24 to be placed into the anchor assembly through a periphery of the assembly, as shown in FIG. 7A. Once the suture or anchor 24 has been received within the respective slots 76, 80, the inner disc 70 may be rotated relative to the outer housing 72, e.g., by engaging disc engagement opening 84 via an instrument, until the suture or tether 24 is retained within housing slot 82 by disc slot 78, as shown in FIGS. 7B and 7C. As the disc 70 is rotated further, the spiraled or curved profile of disc slot 78 (e.g., Fibonacci spiral or curve 60) may urge the suture or tether 24 further against housing slot 82 as the disc 70 depending upon the degree of rotation of disc 70 relative to outer housing 72, as shown in FIGS. 7D and 7E.

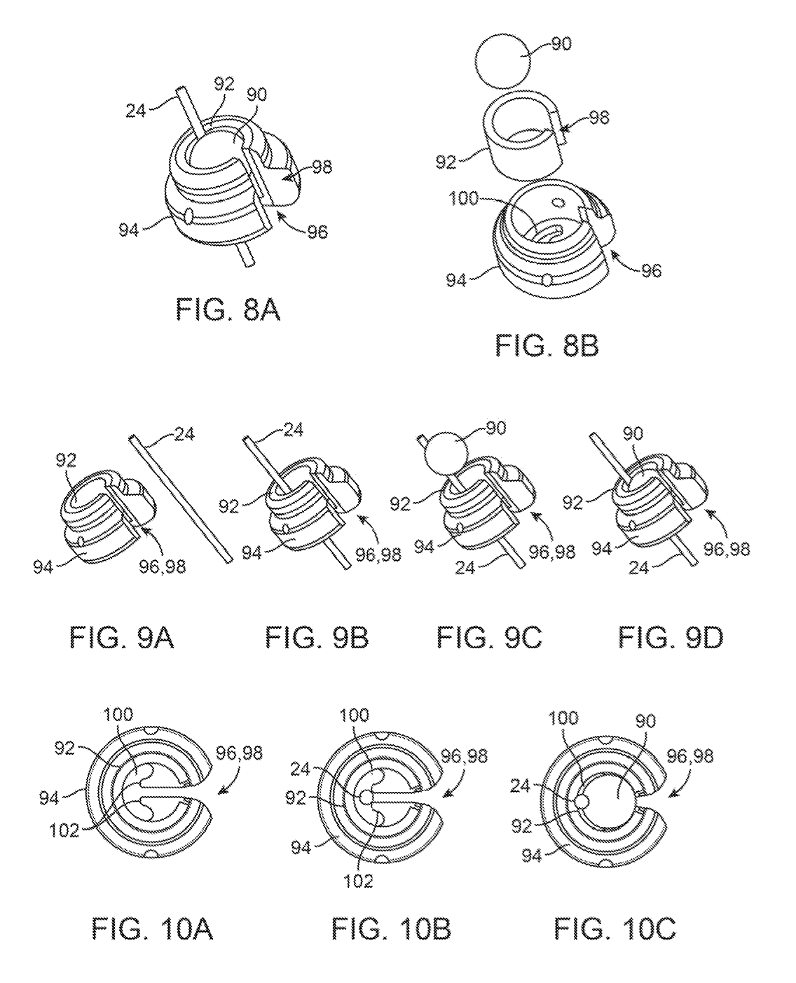

Yet another variation of a locking anchor is shown in the perspective and assembly views of FIGS. 8A and 8B which shows a cam locking mechanism 90 which may accommodate a wide range of suture or tether diameters. In this variation, a compression bearing or ball 90 may wedge the suture or tether 24 against the housing 94, as shown in the perspective views of FIGS. 9A to 9D. A force applied from a direction of the tissue face and opposing cap may preferentially lock the anchor to the suture or tether 24, as shown in the top views of FIGS. 10A to 10C. The locking anchor may be readily unlocked and removed by forcing the anchor assembly to slide relative to the suture or tether 24 towards the tissue.

The cam locking anchor may utilize an assembly having a relatively rigid sleeve 92, outer housing 94, and cam ball 90. The outer housing 94 may be made of a rapidly bioresorbable and highly elastic material to ensure the main body primarily interacting with the surrounding tissue and anatomy may flex as required. This may allow for a larger force distributing footprint without acting as a cast or rigid splinting body. The inner sleeve 92 may be manufactured such that the mechanical characteristics include the rigidity, strength and toughness required to lock the suture or tether 24 against the cam ball 90. In this embodiment, the suture or tether 24 may be introduced into the anchor assembly through a housing opening 96 and sleeve opening 98 which may be aligned relative to one another. The suture or tether 24 may initially slide freely through the anchor assembly through suture thru-hole 100 to allow for adjustment of the anchor positioning relative to the tissue prior to securing the anchor to the suture 24. One or more retaining projections 102 may be optionally defined within the thru-hole 100 to help inhibit the suture or tether 24 from falling out of the housing and sleeve openings 96, 98.

Once the anchor has been desirably positioned along the suture or tether 24 relative to the tissue to be secured, tension may be applied to the suture or tether 24 relative to a grounded sleeve 92 and housing 94 assembly by preferentially applying a moment about the rotational axis of the cam ball 90. As the ball 90 may continue to rotate with the sliding tether until it bottoms out against the inner wall/base of the sleeve 92.

Aside from the variations shown, other alternative embodiments of the locking anchor may include, e.g., a hinge-locking mechanism, where a force applied from the direction of the tissue face and opposing cap may preferentially lock the cap to the tether. Moreover, each of the locking anchors may utilize a side-loading channel for receiving the suture or tether length. This may allow for various sizes or geometries of tissue piercing needles to be used with the suture lengths as the locking anchors may be engaged along the suture lengths proximal to the needle.

Additionally, alternative configurations of the tissue anchors may be used. For example, various geometric shapes that conform to the tissue may be used. In one embodiment that shape will be circular, in others it will be linear, curved, oval, etc. The shape of each implant may create the ideal suturing characteristic for tissue closure, for instance, the circular attachment may create eversion of the tissue while flat exposed areas may spread tension on the circular plate. A flat surface may reduce pain felt by the patient during the degredation of the suture and implant and reduce dehiscence secondary to the direction and diffusion of tension. In other embodiments, the attachment can be linear, creating a sealing of the tissue closure when placed linearly.

The locking anchors may be made of a resorbable or non-resorbable material (e.g., a polymeric material). In certain embodiments, the anchors may be made of a resorbable polymeric material such as a polyester. In other variations, the anchors may made of a resorbable material such as poly(lactic-co-glycolic acid).

While the locking anchors may be made of a biocompatible material, such as a biocompatible polymer, they may be made of a biocompatible and bioresorbable material, e.g., polyester, polyanhydride, polyphosphazene, polyacrylate, polymethacrylate, etc. In other variations, the locking anchors may be made of, e.g., poly(lactic-co-glycolic acid) (PLGA), poly(L-lactic-co-glycolic acid) (90% glycolide:10% L-lactide), etc. Such biocompatible and bioresorbable materials may be absorbed in approximately 1 to 4 weeks during use or approximately 4-6 weeks during use. Other variations may be absorbed approximately 6-8 weeks during use or even longer, such as approximately 2-3 months during use.

In alternative variations, the locking anchors may be comprised of a bioresorbable, synthetic polymeric material, e.g., co-polymer, block polymer, linear polymer, branched polymer, dendritic polymer, cross-linked polymer, etc. In other variations, the polymer may comprise, e.g., polyester, polyurethane, polyvinyl chloride, polyethylene, polyolefin, polyanhydride, polyamide, polycarbonates, polycarbamate, polyacrylate, polymethacrylate, polystyrene, polyurea, polyether, polyalkylether, or polyamine. Exemplary polymers that may be used to make the device include poly(lactic acid), poly(glycolic acid), poly(lactic-co-glycolic acid) (PLGA), poly(anhydride), polyphosphazenes, and poly(caprolactone). In certain embodiments, the polymer is a poly(glycolide-co-lactide) (PLGA). In certain embodiments, the device is made of 50% D,L-lactide and 50% glycolide co-polymer. In certain embodiments, the device is made of 50% L-lactide and 50% glycolide co-polymer. In certain embodiments, the device is made of 85% D,L-lactide and 15% glycolide co-polymer. In certain embodiments, the device is made of 85% L-lactide and 15% glycolide co-polymer. In certain embodiments, the device is made of 90% D,L-lactide and 10% glycolide co-polymer. In certain embodiments, the device is made of 90% L-lactide and 10% glycolide co-polymer. In certain embodiments, the polymer is polyglycolic acid. In certain embodiments, the polymer is poly-β-hydroxybutyrate. In certain embodiments, the polymer is polyacrylic acid ester. In certain embodiments, the device is made of Pebax, Polyimide, Braided Polyimide, Nylon, PVC, Hytrel, HDPE, or PEEK. In certain embodiments, the device is made of a fluorinated polymer such as PTFE, PFA, FEP, and EPTFE. In certain embodiments, the device is made of latex. In certain embodiments, the device is made of a natural polymer. In certain embodiments, the natural polymer is a polysaccharide such as cellulose or derivatives thereof. In certain embodiments, the natural polymer is a protein. The locking anchors may be made of a material that is bioabsorbed after the device is no longer needed (e.g., after the tissues have healed). For example, the device may degrade in vivo after 1 week, 2 weeks, 3 weeks, 1 month, 2 months, 3 months, 4 months, 5 months, 6 months, etc. In certain embodiments, the device is designed to degrade after approximately 4-6 weeks in vivo.

In certain embodiments, the fastening device may also be constructed of a shape memory plastic or metallic material. Such shape memory plastic or metallic materials can return to some previously defined shape or size when subjected to the appropriate thermal procedure or similar form of energy. That is, shape memory alloys or plastics can be plastically deformed at some relatively low temperature and, upon exposure to some higher temperature, will return to their original shape. Metal materials that exhibit the shape memory effect include a number of copper allow systems and the alloys of gold-cadmium, nickel-aluminum, and iron-platinum. In addition, plastic materials that exhibit the shape memory effect have been developed and include, for example, materials disclosed in U.S. Pat. Nos. 6,720,402, 6,388,043, 6,160,084 (each of which is incorporated herein by reference in its entirety).

In certain embodiments, the fastening device may be provided in a shape that is easily inserted into the mucosa. Thermal energy may then be applied to cause the fastening device to change shape (e.g., make the fastening device close together), thus providing a rapid closure of the fastener through the mucosal flaps with merely an application of heat or energy. In certain embodiments, the fastening device may comprise an elastomeric material, including for example, a polyhydroxyalkanoate (PHA) material. In certain embodiments, the fastening device may comprise a crystalline or amorphous polymer combined with an elastomeric polymer. For example, a highly crystalline polylactide may be blended with a polyhydroxybutarate; specifically 80-97% Poly-L-Lactides (PLLA) and 20-3% PHA in certain embodiments. Similarly, caprolactone or trimethyl carbonate may be added to a crystalline polymer to make it more elastic. Elasticity of the construct can be achieved through the addition of the caprolactone or trimethyl carbonate to a lactide or glycolide monomer since the caprolactone and trimethyl carbonate have relatively low melting temperatures (e.g., 60° C. tier caprolactone).

Such material can provide an elasticity that is similar to that of the surrounding tissue. By providing a similar elasticity to the surrounding material, the fastening device can flex with the surrounding tissue. This can increase the ability of the fastening device to maintain approximation of the tissue during healing, and can reduce the likelihood that the fastening device will tear or otherwise damage the surrounding tissue.

Moreover, the locking anchors and/or sutures may be optionally coated with one or more various agents. For instance, examples of coatings may include a timed release formulation of a pharmaceutical agent such as a hemostatic agent, anti-inflammatory agent, a steroid, antibiotic, anesthetics, pain reliever, hemostatic agent, etc. The agent may be released over hours, to days, to weeks, to months. In certain embodiments, the coating is a polymeric coating impregnated with a therapeutic agent. Classes of therapeutic agents that may be delivered by the device include DNA, RNA, nucleic acids, proteins, peptides, or small molecules. Exemplary therapeutic agents include antibiotics, anti-inflammatory agents, corticosteroids, vasoconstrictors, vasodilators, coagulants, pain relievers, etc. The coating may also include a radioopaque agent for imaging of the device.

In other variations, the devices may be made of a non-biodegradable material or otherwise coated with, e.g., Teflon (PTFE), hyaluronidate, polymers, polysaccharide such as hyaluronate, etc. The locking anchors may also be coated to make the device more biocompatible. Many coatings for medical devices are known in the art and may be applied to the inventive fastening devices.

Although custom tether materials and sizes may be housed within the deployment instrument for automatic deployment, the anchors and instruments herein may be compatible with multiple conventional sutures available in the operating room due to the locking mechanisms employed within the entry and exit cap assemblies and the side loading nature of the anchors.

Additionally, the locking nature of the anchors may allow for the adjustment of tension on the suture and similarly may allow for adjustment of the length of the tether or suture between the locking cap assemblies as well as the position of the needle exit and entry sites relative to the tissue flap edges. This may provide for the physician to adjust the tissue fastening site mechanics relative to the spacing between tissue flaps associated with the amount of tissue excised. This also allows the physician to adjust the cap and tether deployment relative to the types of tissue and healing characteristics at the target site. Alternatively, many staple or auto-knot tying devices include a fixed throw of a piercing needle or fixed length of suture or tether between tissue engaging components. The fixed aspect of these devices equates to significant variance in the amount of tension applied to the target tissues when closing wounds with different gap dimensions.

Because of the adjustability of the locking caps and delivery instruments, the physician may use both tactile and visual feedback to close wounds. This may enable the physician to alter their approach based on surgical experience.

Deployment Systems

Deployment instruments for holding and applying the various locking anchors to close or approximate two or more tissues are also provided. Such instruments may include a space for holding a plurality of fastening devices that may be needed in a given surgery or procedure. The instrument may include a handle for comfortable gripping of the instrument and may work by applying the fastening anchors to the mucosa or other tissues to be joined and fastening the device. The deployment instrument may be disposable or suitable for sterilization and re-use. The delivery system may include a combination of disposable and reusable components. For example, in one variation, the cartridge of caps and reel of suture may be sterilized and packaged from a reusable or disposable delivery instrument including, e.g., a handle, actuator, etc.

The deployment instrument may optionally include an energy source within the handle, e.g., battery supply, or may be in communication with an external energy source such as a radio-frequency generator to deliver energy that may be used to actuate, deform, or assemble components of the cap and tether configuration.

Generally, the deployment instrument may (1) lock an initial entry cap onto a tether and needle assembly (2) assist in the initial pass of the needle portion of the assembly through the first tissue flap (3) assist in passing the needle through the second tissue flap (4) engage the exit side suture with the distal assembly, including at least one locking cap (5) cinch the exit locking cap against the tissue with sufficient force to mate the two edges/flaps of the wound (6) lock the exit cap to the exit side tether (7) lock the next entry cap to the needle and tether assembly extending from proximal face of the first exit cap (e.g., simultaneous to (6) locking the exit cap to the exit side tether) (8) cut the exit tail of the tether between the locked exit cap and next locked entry cap to complete the first wound closure and prepare the second needle/tether/entry cap assembly for the second wound closure site. Moreover, the deployment instrument may mate with a disposable cartridge having, e.g., at least 10 but nominally 30 locking anchor assemblies to provide at least 5 but nominally 15 total wound closure sites per procedure. Although other variations may incorporate few or additional locking anchor assemblies.

A piercing needle may be omitted from the deployment instrument and furthermore, the deployment instrument does not require any piercing element to pass through the instrument as having a curved or large diameter needle would potentially require a relatively larger deployment instrument. Moreover, the deployment instrument may utilize a side-loading channel much like the locking anchors thus enabling the instrument to engage exposed suture at any point along its length. This may further allow the deployment instrument to engage close to the target tissue closure site potentially inside a body cavity and/or after exposed from a distal end of laparoscopic tooling. This may also allow for the deployment instrument to engage suture at a distance from the target tissue closure site where visualization of a suture tail may be easier such as from the mouth, nose, or surgical created body cavity. Because a single instrument provides multiple functions (e.g., needle/tissue pick up, closure cap attachment, suture-trimming, etc.), the deployment instrument may mate with other laparoscopic tool lumens.

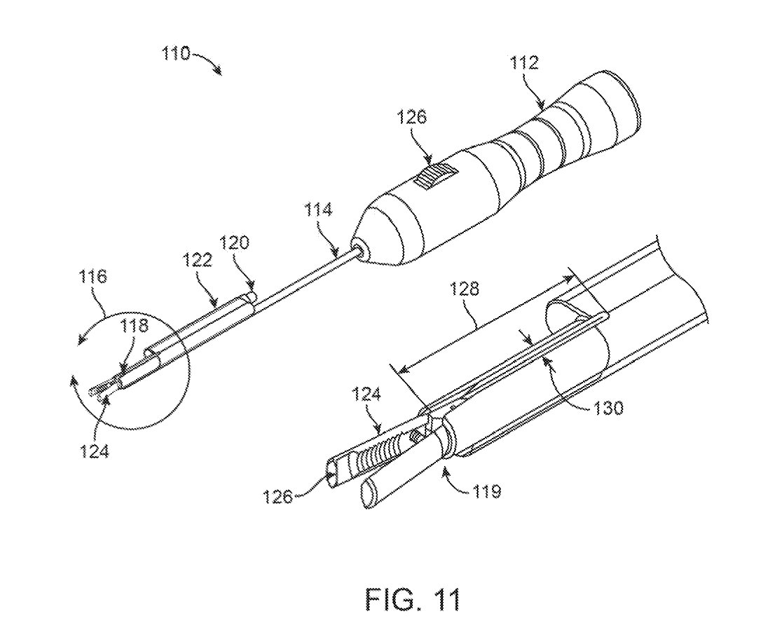

An example of one variation of the deployment instrument is shown in the perspective view of FIG. 11. The deployment instrument 110 may apply an axial compression force to the locking anchor assembly described herein. Alternative locking anchor designs may utilize alternative mechanisms at the distal end of the delivery tool to actuate/lock the entry and exit caps to the tether. However, the primary sequence of events and methods disclosed here would apply to any locking anchor and tether closure assembly disclosed herein.

The deployment instrument 110 may generally comprise a proximal handle 112, main shaft 114 attached to the handle 112, and distal assembly 116 positioned at a distal end of the main shaft 114. The proximal handle 112 may include ergonomic features to comfortably fit in one hand of the user and mechanical actuators to translate proximal motion to distal motion as used by the locking anchors and needle gripper. The distal assembly 116 may also incorporate a main distal housing 118 which defines a distal housing opening 119 as shown in the detail view of distal assembly 116. A locking cap cartridge 120 may also be attached to the main shaft 114 and/or distal housing 118 and may contain the locking anchors for deployment into or against the tissue. A locking cap staging housing 122 may also be defined within the distal housing 118 where the anchors may be chambered into the distal housing 118 for loading of the suture or tether for deployment into the tissue.

Optionally, a gripper tool 124, such as forceps or graspers, may be actuatable and slidably disposed through the distal housing 118 and adjacent to the locking cap cartridge 120. The gripper tool 124 may define one or more cutting edges or blades 126 for selectively severing the suture or tether, and it may also define a surface for grasping or manipulating a needle and/or the suture or tether as well. Moreover, the distal housing 118 (through which the gripper tool 124 may translate) may further define a suture slot having a length 128 and width 130 which extends longitudinally along the distal housing 118 for receiving the suture or tether into the locking anchors prior to or during deployment, as further described herein.

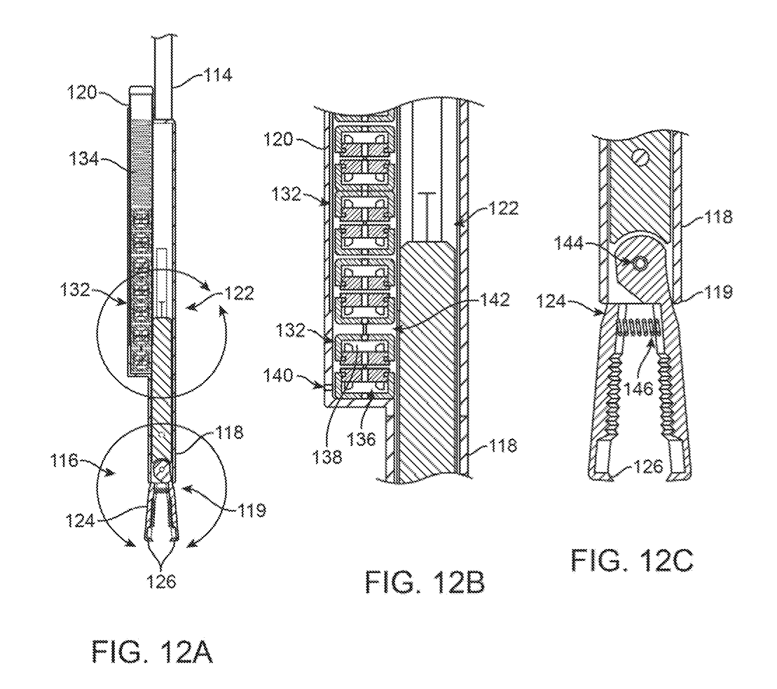

The optional needle gripper 124 and the locking cap cartridge 120 are shown in the cross-sectional side views of FIG. 12A to 12C. As illustrated in this variation of the deployment instrument 110, the gripper 124 may be slidably contained within the distal housing 118 of the distal assembly 116 and may be utilized as a needle pick up similar to conventional surgical tooling as well as an inner plunging tool for controlling the position and state of the locking anchors. As a plunging tool, the needle gripper 124 may apply the compression locking force to the locking entry and exit anchors. The gripping tool 124 may also incorporate the suture or tether cutting element 126 at the distal most point of its claws used to cut the trailing/exit end of the suture or tether upon completion of each wound closing step.

The distal housing 118 may define the suture slot 128 through its outer wall that aligns with the mouth and slot of the locking anchor assemblies during the tether engagement step of the closure process. This slot 128 may be wide enough to easily accept the suture or tether element and long enough to ensure the suture or tether fully engages at least two locking anchor assemblies (exit and entry). The assembly may also include an anchor staging housing 122 mounted off axis from the main body assembly and may further include a proximal mating feature to accept a locking anchor cartridge 120. This cartridge 120 may contain multiple locking anchor pairs 132, e.g., an entry 136 and exit 138 anchor pair, and may be packaged separately from the tool, as shown in FIGS. 12A and 12B. The cartridge 120 may include as few as 5 and as many as locking anchor pairs 132. In one embodiment the separately packaged cartridge 120 and anchors may be disposable and configured to mate with the reusable delivery device main body.

During use, the anchor pairs 132 may be ejected from the cartridge 120 via a cartridge housing spring tab 140 located along the cartridge 120 in proximity to the anchor staging housing 122 which may push or slide the anchor pairs from the cartridge 120, through a cartridge opening 142 defined between the cartridge 120 and distal housing 118, and into the distal housing 118 for loading and deployment into or against the tissue, as described in further detail below. The next anchor pair 132 may be pushed or urged distally through the cartridge 120 via a biasing element 134, such as a spring, into position adjacent to the distal housing 118.

Manipulation of the optional gripper tool 124 includes advancement and retraction of the claws relative to the distal housing opening 119. These claws may be urged open by, e.g., a biasing element 146 such as a spring, positioned near the pivot point at their base. The claws may spring hinge about pivot 144 to open when they are advanced beyond the distal housing opening 119 and are no longer constrained within the housing inner diameter. When retracted, the distal face inner edge of the housing may interface with the ramping proximal face 148 of the claws to force them closed. The position of this interface relative to the claw pivot 144 may be modified to increase or decrease the claw closing force relative to the tensile force applied at the handle by the user. The severity of the angle of this proximal ramping face 148 of the claws 124 may be modified to change the amount of longitudinal motion required to apply the range of gripping forces and the coarse or fine adjustment capabilities for this gripping feature of the instrument.

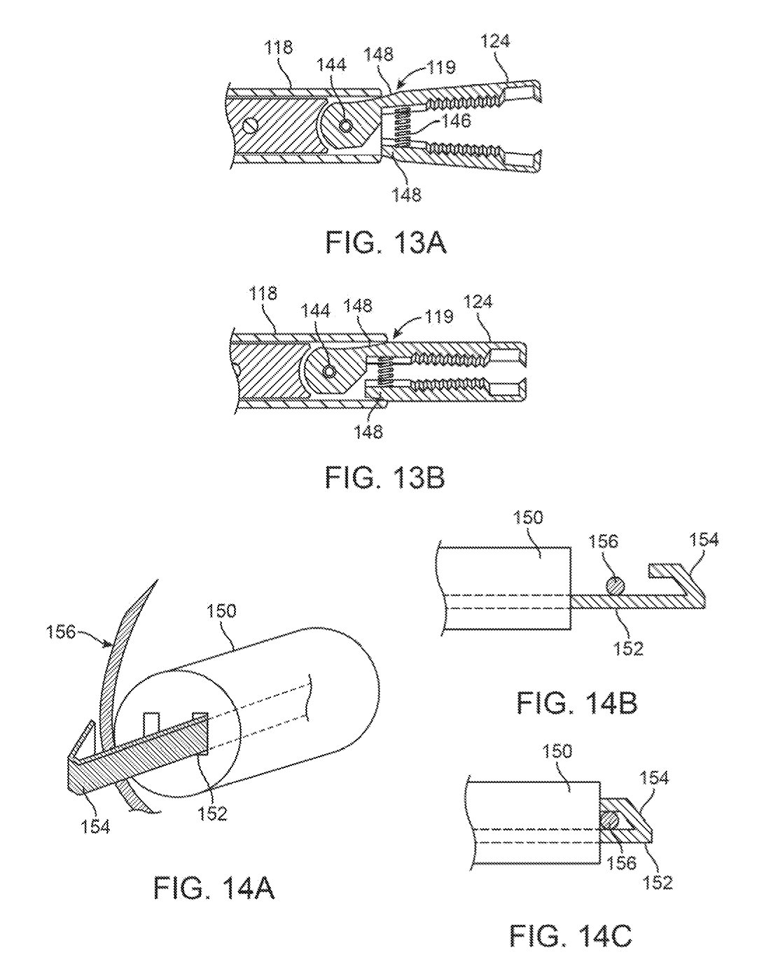

FIGS. 13A and 13B show detail cross-sectional side views of an alternative gripper configuration which takes advantage of a telescoping motion between the inner components and an outer sheath. Both configurations may be easily incorporated into the deployment instrument herein.

FIG. 14A shows a perspective view of another alternative gripper configuration. This variation illustrates a hook grabber configuration which may utilize a bent arm 154 extending from the retraction arm 152 to hook needle 156. The arm 154 may retract to trap the needle 156 between the inner bend radius of the bent arm 154 and face of the main body 150. FIGS. 14B and 14C illustrates a side view of how the bent arm 154 may be projected or retracted to capture the needle 156 or suture 24.

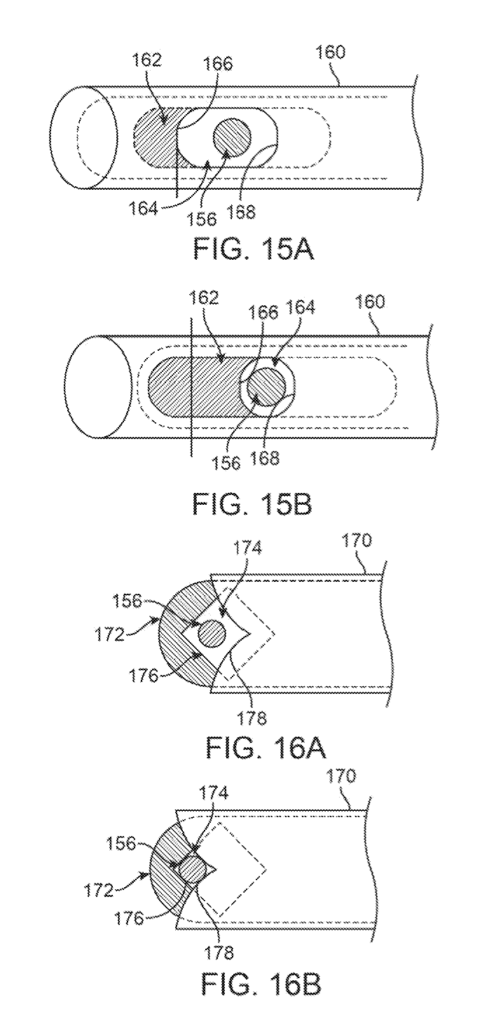

FIGS. 15A and 15B show side views of yet another variation of a gripper which utilizes an inner mandrel 162 which may include a through-hole 164 sufficiently large enough to easily accept the needle 156. The main body 160 may include a mating hole that allows for full communication through the instrument when aligned. In use, the needle 156 and/or suture 24 may be passed into or through the through-hole 164 and the inner mandrel 162 may be retracted relative to main body 160 to trap the needle 156 (or suture) between the mandrel proximal face 166 and main body distal surface 168. The retraction of the mandrel 162 may be varied to accommodate various size needles and/or sutures lengths.

FIGS. 16A and 16B show side views of yet another variation where either variation may include a “V”-shaped groove at the mandrel proximal face 176 to promote increased grip on the needle 156. With the through-hole 174 aligned, the needle 156 may be passed through the instrument. The inner mandrel 172 may be retracted relative to the main body 170 to lock the needle 156 between the mandrel proximal face 176 and main body distal surface 178.

With this or any of the needle/suture manipulation instruments variations, any of the embodiments may be optionally incorporated with the deployment instrument 110 in various combinations.

The deployment instrument may also incorporate an optionally removable multiple anchor cartridge 120 which allows for rapid deployment of interrupted closures along a wound. As described, the deployment instrument may house two or more locking anchor assemblies that may serve as an entry and exit cap at surfaces of the opposing wound tissue flaps. These anchors may be deployed to the target site at a distance ranging from about, e.g., 6 to 12 inches, for UPPP like procedures or using a flexible main shaft and deployed through an endoscope or laparoscopic ports requiring longer and smaller diameter tooling.

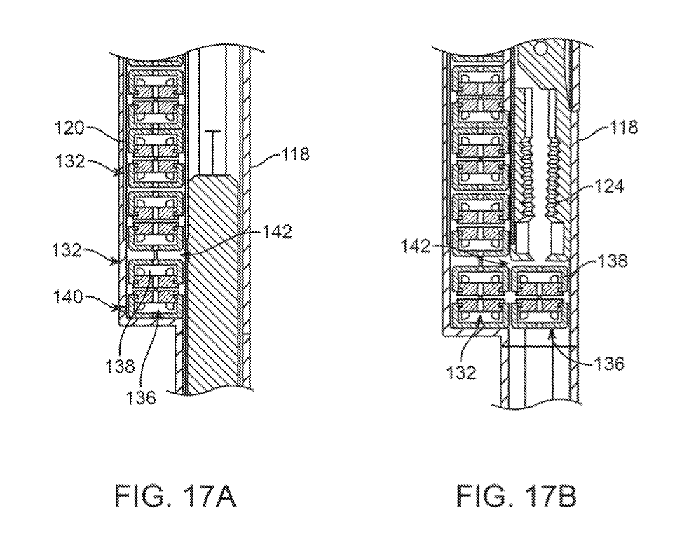

FIGS. 17A and 17B illustrate cross-sectional side views of one variation of an anchor cartridge 120 which is configured to allow for a pair 132 of locking anchors which are stacked or aligned with one another within the cartridge 120 to move from the staging housing to the main body. If the optional needle gripper 124 is used, the needle gripper 124 may be positioned in its distal working position such that its proximal body fills the annular space in the main housing 118 preventing the locking anchors from sliding sideways. When the gripper 124 is fully retracted using the controls on the proximal handle, the spring tab is free to push an exit/entry anchor assembly laterally through cartridge opening 142 and into the main housing 118 distal to the claws of the gripper 124, as shown. Simultaneously, the anchor cartridge compression spring 134 may slide the remaining anchors 132 distally staging the next exit/entry anchor pair.

Once the exit/entry anchor pair 132 is positioned distal to the gripper 124 in the main housing 118, it may be pushed forward until flush with the distal opening 119 of the main housing 118. With the exit/entry anchor pair 132 in the distal position, the deployment instrument 110 may be configured to engage the needle and suture or tether near the needle exit point from the tissue.

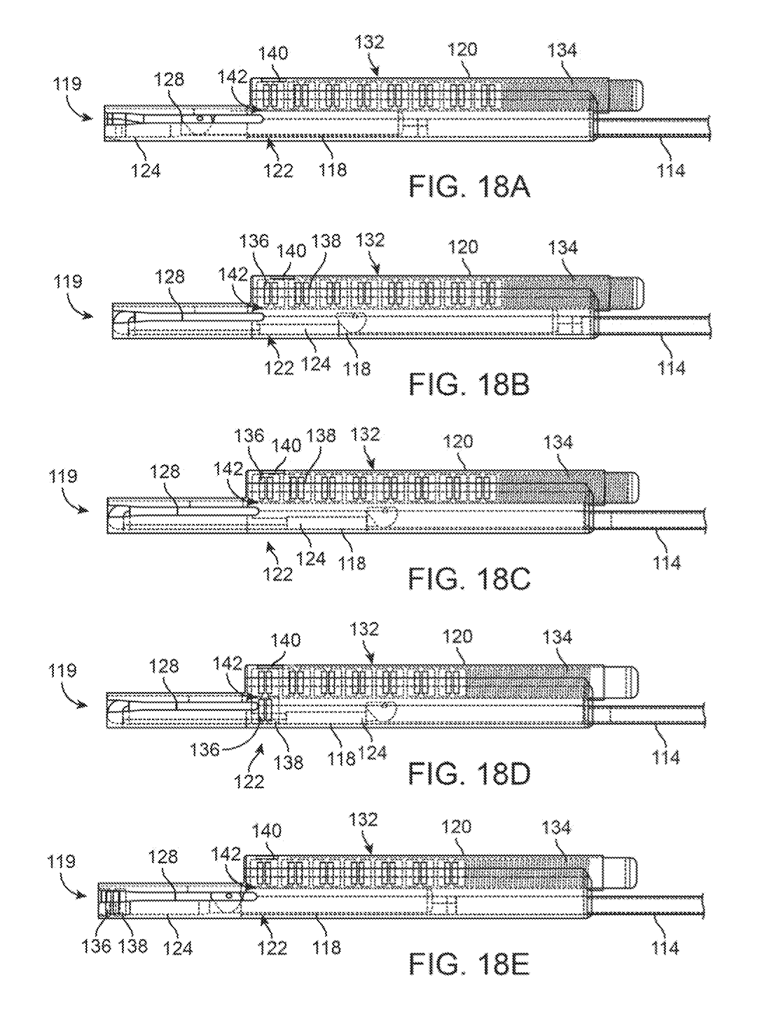

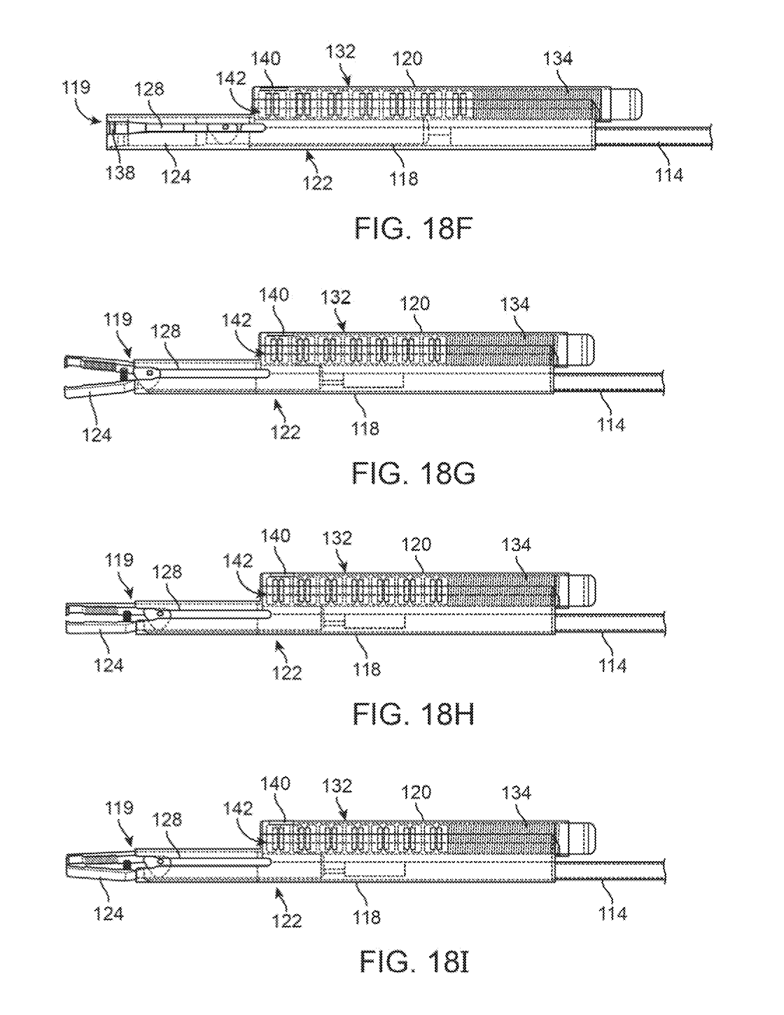

As shown in the partial cross-sectional side views of FIGS. 18A to 18I, the distal detail of the deployment instrument 110 and position of the locking anchors 132 and gripper 124 as anchor components are shuttled from the staging position 122 to the distal main housing 118 ready to accept the tissue exiting suture or tether and needle. The anchor cartridge 120 and instrument housing are shown along with the gripper 124 in its home position at a distal position within the main housing 118, as shown in FIG. 18A. FIG. 18B shows the gripper 124 retracted proximally in its second home position proximal within the main housing 118 and adjacent to the staging area 122. FIG. 18C shows the gripper 124 fully retracted opening a space in the main housing 118 for receiving the locking anchor pair 132.

FIG. 18D shows a locking anchor pair 132 pushed laterally via cartridge housing spring tab 140 into the main housing 118. FIG. 18E shows the gripper 124 pushing the locking anchor pair 132 to a distal position through the main housing 118. FIG. 18F shows the gripper 124 compressing the locking anchor pair 132 to lock both exit 136 and entry 138 anchor to an axially positioned suture or tether which may be introduced through the longitudinal suture slot 128 defined along the distal portion of main housing 118. FIG. 18G shows the gripper 124 claws in an open position, e.g., for grasping or manipulating a needle or suture length and FIG. 18H shows the gripper claws in its closed position. Finally, FIG. 18I shows the gripper 124 claws in tether cutting position to release the anchor pair 132 deployed within the tissue.

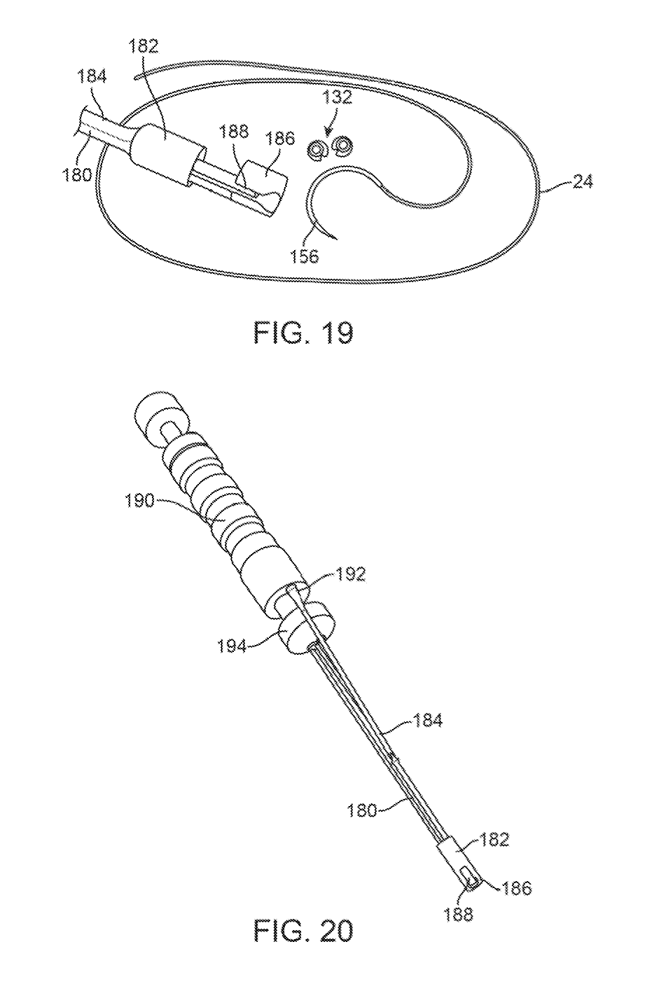

FIG. 19 shows an assembly view of another variation of a deployment instrument (which may optionally omit the anchor cartridge) having a shaft 180 with a locking anchor staging housing 186 attached at a distal end at also attached to a stationary handle 194 at its proximal end. FIG. 20 also shows a perspective view of the deployment instrument. A suture slot 188 may be defined longitudinally along a length of the staging housing 186 for receiving a suture length into an anchor for deployment. An adjacent actuation shaft 184 attached at its distal end to a housing 182 may also be attached 192 to an actuation handle 190 at its proximal end where the actuation handle 190 may be translated relative to the stationary handle 194. Also illustrated is a pair of locking anchors 132 and a suture length 24 attached to a suturing needle 156 as part of the assembly prior to deployment in the tissue region.

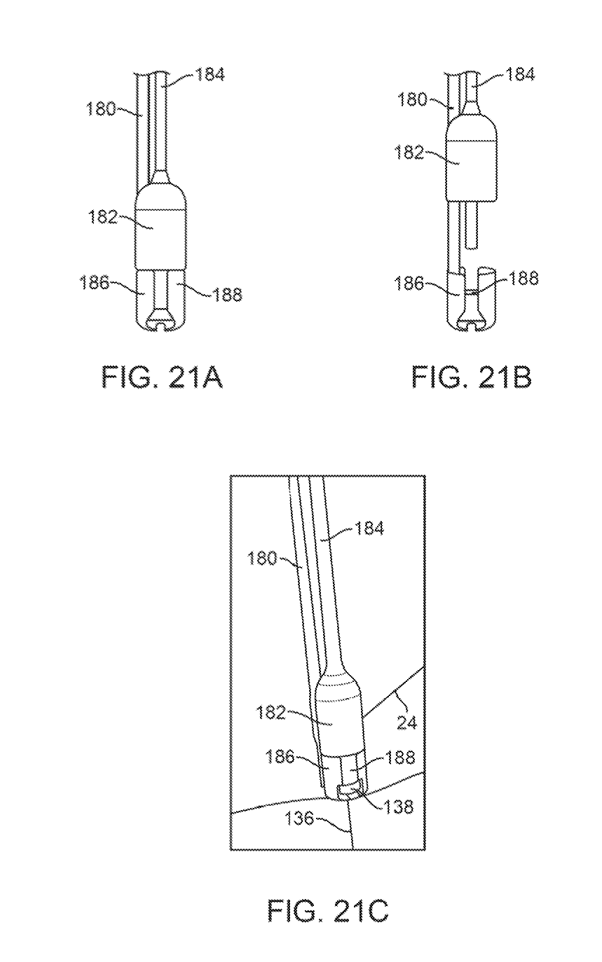

FIGS. 21A to 21C show detail side and perspective views of the deployment instrument of FIG. 19 illustrating how a plunging portion (e.g., housing 182) may be moved relative to an anvil portion (e.g., anchor staging housing 186) to secure an anchor to the tissue region. In an example of use, one or more of the anchors such as anchor 138 may be placed within anchor staging housing 186 with the open slot of the anchor aligned with the suture slot 188. A length of suture or tether 24 may be introduced through suture slot 188 as well as within the open slot of the anchor positioned within anchor staging housing 186 and the actuation shaft 184 may be actuated to close housing 182 relative to anchor staging housing 186, as shown in FIG. 21C. Actuating the assembly may secure or crimp the anchor upon the suture or tether 24 for placement upon or against the tissue for approximating the tissue regions.

Closure Methods

In another aspect methods of using the fastening devices and/or the instruments are provided. The devices and instruments may be used in a variety of surgeries or procedures. For example, the surgeries or procedures may involve the approximation of mucosal surfaces of the head and neck. Other procedures may involve surgeries or procedures involving the oral cavity, oropharynx, hypopharynx, throat, or laryngeal surfaces such as UPPP procedures, tonsillectomies, etc.

For example, the apparatus and methods herein may be used to close the tonsillar pillars. Sealants such as a fibrin-based products, chitosan-based products, thrombin-based products, alpha-cellulose based products, collagen-based products, albumin-based products, etc. can be used in conjunction with the fastening devices in order to reduce pain, reduce bleeding, and/or otherwise improve the outcome. In alternative procedures, coatings, such as protein or growth factor based products, can be used in conjunction with the fastening devices in order to enhance healing and/or otherwise improve the outcome. For instance, tonsillectomy procedures may be performed with the devices and apparatus herein along with a sealant placed on the tonsillar bed.

Other procedures or applications for these devices and methods may include, e.g., removal of a tumor of the head or neck, dental or oral surgery, closing of laryngeal or pharyngeal defects, procedures on the gastrointestinal tract and genitourinary system, neurosurgery procedures such as dural closure, etc.

The devices described herein may be optionally embodied in a kit. For instance, such a kit may include one or more sizes of the locking anchors, pharmaceutical agents (e.g., anesthetics, antibiotics, etc.), a deployment instrument for applying/fastening the locking anchors, a separate instrument for removing the anchors, instructions for use, etc. Moreover, the items may be sterilely packaged for the use by a treating physician.

Generally in use for closing a wound or approximating two regions of tissue towards one another, a force distributing anchor may be attached to a suture length having an attached needle. The suture and a first anchor assembly may be passed through a first tissue flap of the target closure site until the first anchor is flush with the needle entry point through the tissue. A second and third locking anchor may be simultaneously locked to the suture such that the second anchor is flush with the tissue at the needle exit site along a second tissue flap while the first and second tissue flaps are approximated towards one another along the suture and placed into contact. Simultaneously and/or subsequently, the suture between the second anchor and the third anchor may be severed (automatically by the deployment instrument or manually by the physician) to prepare the third anchor for a subsequent tissue closure.

The second anchor or the deployment instrument may be used by the physician to control the amount of tension applied to the tether when approximating and/or securing the tissue to one another. Optionally, the deployment instrument may be calibrated to automatically lock the second anchor to the suture when a predetermined amount of force is applied. The deployment instrument may also attach the second anchor to the suture and simultaneously sever the suture when it exits the second anchor. Additionally, the deployment instrument may also be optionally calibrated to attach the second anchor to the suture when a predetermined tension has been achieved and/or simultaneously sever the suture where it exits the second anchor when placed against the secured tissue.

In approximating and secure two apposed tissue regions, the anchor pairs may be deployed and secured with the deployment instrument, e.g., repeated at least 3 times, 5 times, or 10 times, or more. In certain procedures, the steps are performed in the context of a uvulopalatopharyngoplasty (UPPP) with or without tonsillectomy procedure. In other procedures, the steps are performed in the context of a uvulopalatal flap (UPF) procedure. In other procedures, the steps are performed in the context of removal of a tumor. In still other procedures, the steps are performed in the context of closing a laryngeal or pharyngeal defect. In still other procedures, the steps are performed in the context of closing an Eustachian tube orifice. In certain procedures, the steps are performed in the context of a dental procedure. In certain procedures, the steps are performed for laparoscopic bowel surgery, cholesystectomy, endoscopic gastric surgery or orthopedic soft tissue surgery, or interventional gastroenterology procedures involving manipulating an instrument through the working channel of an endoscope. In specific procedures, the method further comprises applying a tissue sealant to the approximated tissues. In certain procedures, the tissue sealant is a fibrin-based sealant, while in other procedures, the tissue sealant is a chitosan based product, thrombin based product, alpha cellulose based product, collagen based product, or albumin based product.

Although suitable for use in closing or approximating mucosal tissues, the fastening devices may be used in other non-mucosal tissues, e.g., approximating a mucosal surface to a non-mucosal surface; approximating a non-mucosal surface to a non-mucosal surface, etc.

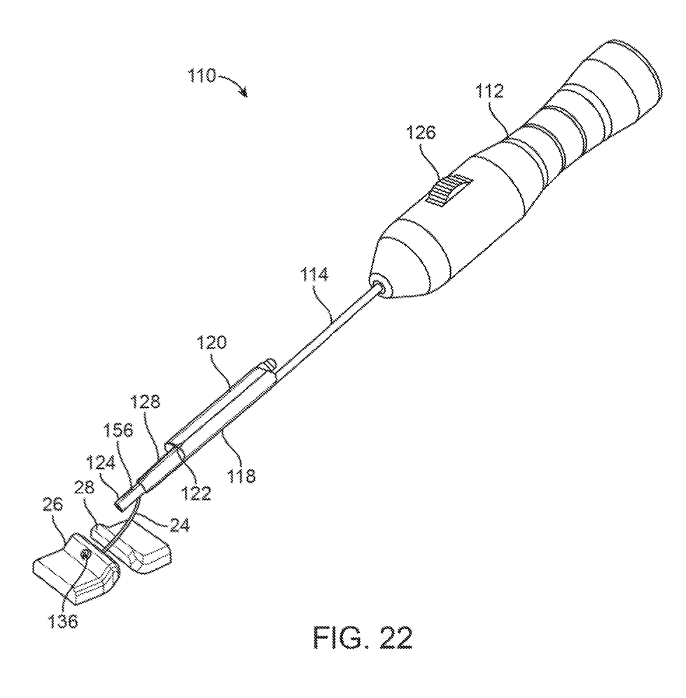

Turning now to FIG. 22, a perspective view of a variation of the deployment instrument 110 is shown illustrating how a suture or tether length 24 attached to a deployed locking anchor 136 secured to a first portion of tissue 26 may be attached to the deployment instrument 110 by having the deployment instrument 110 grab the needle 156 via grabbing jaws 124 as it exits the first portion of tissue 26.

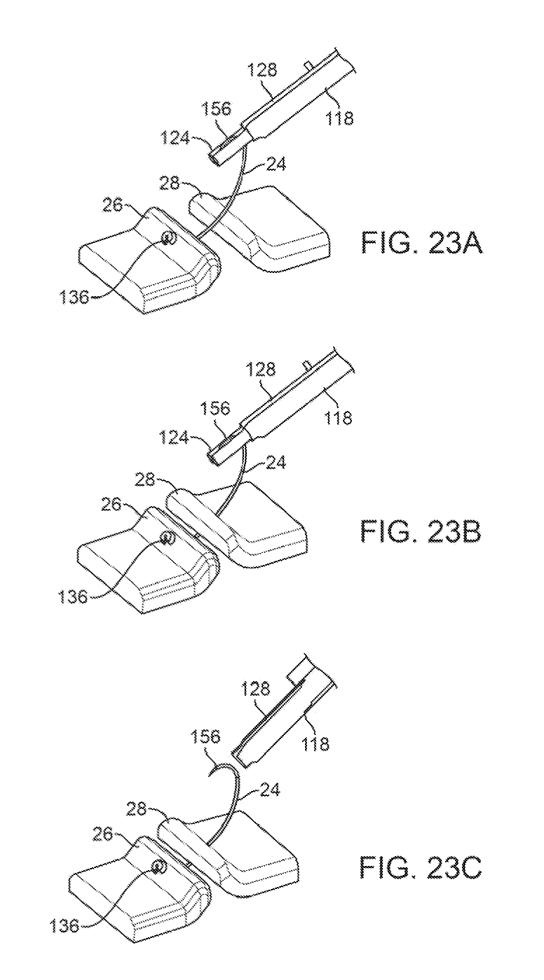

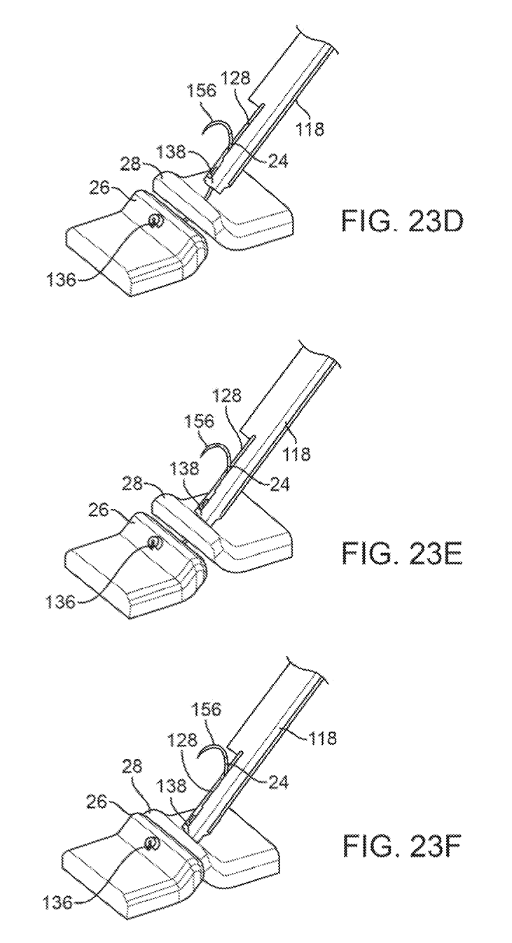

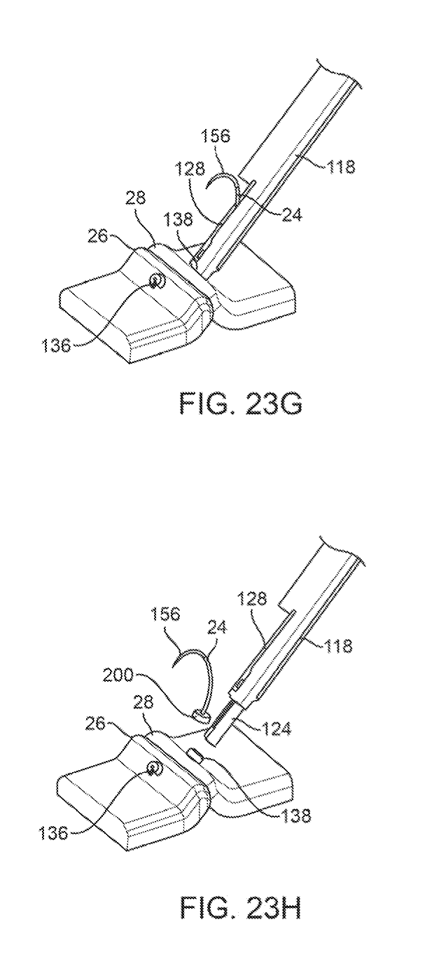

FIGS. 23A to 23H illustrate how a first locking anchor 136 may be secured to the first portion of tissue 26 and tightened to a second corresponding locking anchor 138 secured to a second portion of tissue 28 via the deployment instrument 110 and subsequently tightened to approximate the first 26 and second portions 28 of tissue.

FIG. 23A shows how the first locking anchor 136 may be pulled to sit flush against the first tissue portion 26. The deployment instrument 110 may be positioned to grab and control the needle 156 as it exits the first tissue flap 26 if a grasper 124 is used with the deployment instrument 110. Otherwise, the physician (or assistant) may manually grasp the needle 156 and apply the suitable tension. As shown in FIGS. 23B and 23C, the needle 156 grasped and engaged by the grasper 124 (or manually via another instrument) may be passed through a second tissue flap 28 to be approximated with the first tissue flap 26.

Once the suture or tether 24 has been passed through both tissue portions 26, 28 to be approximated or affixed, the suture or tether 24 may then be placed against along suture slot 128 of main housing 118 and loaded along a side opening slot of at least a second anchor 138 cycled within the main housing 118, as shown in FIGS. 23D and 23E. Optionally, a third anchor may also be cycled within the deployment instrument to load the subsequent anchor into the distal tether engagement position for the next anchor deployment.

FIG. 23F illustrates how with the needle 156 and exit suture or tether 24 in one hand, the physician (or assistant) may engage the exit suture or tether 24 close to its exit from the second tissue flap 28. The suture or tether 24 may slide into the slot 128 in the tool main housing 118 and continue the engagement with the tool 110 until concentric with the next locking anchor pair. The deployment instrument 110 may be pushed toward the target tissue while tension is maintained on the exit suture or tether 24 and needle 156, as shown in FIG. 23G. This causes the deployment instrument 110 to slide down the exit suture or tether 24, contact the exit tissue flap 28, and cinch the tissue flaps 26, 28 to one another. Using the proximal handle controls, the physician (or assistant) advances the deployment instrument needle grabber 124 assembly and compresses the exit and entry anchors against the tissue surface. This compressive force snaps both anchors 136, 138 closed fixing the exit suture or tether 24 to both anchors 136, 138. The tissue flaps 26, 28 are fixed together at this closure site.

FIG. 23H shows how the deployment instrument 110 may then be disengaged from the locked exit and entry anchors 136, 138, suture or tether 24 and needle 156. The physician may apply tension to the needle/tether and advance the needle grabber assembly 124 from the distal end of the deployment instrument 110. The distal pinchers of the grabber jaws 124 may be used to cut the suture or tether 24 between the exit and entry anchors 136, 138 creating the next entry anchor 200, needle 156, and suture or tether 24 assembly.

The applications of the disclosed invention discussed above are not limited to certain treatments or regions of the body, but may include any number of other treatments and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A fastening device, comprising:
a housing having one or more adjustable tissue anchors aligned longitudinally, wherein each of the one or more adjustable tissue anchors defines a receiving channel along a periphery of the tissue anchors for receiving a tether; and
a plunger translatable relative to the housing, where movement of the plunger urges the one or more tissue anchors to attach securely to the tether.

2. The fastening device of claim 1, wherein the tether has a length which is positionable within the receiving channel of each of the tissue anchors.

3. The fastening device of claim 2, wherein the tether comprises a suture.

4. The fastening device of claim 1, wherein the housing comprises a cartridge having a plurality of tissue anchors aligned therein.

5. The fastening device of claim 1, wherein the housing defines an opening therealong for receiving the tether.

6. The fastening device of claim 1, wherein the plunger comprises a grasper.

7. The fastening device of claim 1, further comprising a handle extending from a proximal end of the housing.

8. The fastening device of claim 1, wherein the tissue anchor comprises a first portion which defines a first receiving channel and a second portion which defines a second receiving channel, the first portion being rotatable relative to the second portion from an open position where the first and second receiving channels are aligned to a locking position where the first receiving channel and second receiving channel are misaligned.

9. The fastening device of claim 8, wherein the first receiving channel defines a spiraled or curved profile.

10. The fastening device of claim 1, wherein the tissue anchor comprises a cam mechanism.

11. The fastening device of claim 10, wherein the cam mechanism comprises a collar body and an insert positionable within the collar body and a bearing slidably positionable within the insert.

12. The fastening device of claim 1, wherein the tissue anchors are made of a biocompatible and/or bioresorbable material.

13. The fastening device of claim 1, wherein the tissue anchors are coated with an agent.

14. A method of fastening tissue in a procedure, comprising:
passing a tether through a first portion of tissue until a first anchor attached to the tether is placed against the first portion of tissue;
passing the tether through a second portion of tissue;
aligning a second anchor with the tether;
tensioning the first anchor and the second anchor relative to one another such that the first and second portions of tissue are approximated towards one another; and,
securing the second anchor to the tether such that the first and second portions of tissue remain secured to one another.

15. The method of claim 14, wherein passing a tether comprises passing a needle having a length of suture attached through the first portion of tissue.

16. The method of claim 14, further comprising attaching the tether to the first anchor along a receiving channel defined along a side periphery of the first anchor prior to passing a tether through a first portion.

17. The method of claim 14, wherein passing the tether through a second portion comprises passing a needle having a length of suture attached through the second portion of tissue.

18. The method of claim 14, wherein aligning a second anchor comprises introducing the tether through a receiving channel defined along a periphery of the second anchor.

19. The method of claim 18, wherein the second anchor is positioned within a deployment instrument.

20. The method of claim 14, wherein tensioning the first anchor and the second anchor comprises tensioning the tether through the second anchor positioned within a deployment instrument.

21. The method of claim 14, wherein securing the second anchor to the tether further comprises securing a third anchor to the tether.

22. The method of claim 14, further comprising cutting the tether from the second anchor.

23. The method of claim 14, wherein the procedure comprises an uvulopalatopharyngoplasty (UPPP) procedure.

24. The method of claim 23, wherein the procedure further comprises a tonsillectomy procedure.

25. The method of claim 14, wherein the procedure comprises a tonsillectomy procedure.

26. The method of claim 14, wherein the procedure comprises an uvulopalatal flap (UPF) procedure.

27. The method of claim 14, wherein the procedure comprises a gastric tissue approximation procedure.

28. The method of claim 14, wherein the procedure comprises an intestinal tissue approximation procedure.

29. The method of claim 14, wherein the procedure comprises a tendon approximation procedure.

30. The method of claim 14, wherein the procedure comprises a ligament approximation procedure.
